(19) United States
(12) Patent Application Publication
(10) Pub. No.: US 2005/0053670 A1
(43) Pub. Date: Mar. 10, 2005

(54) Composition for easing human childbirth

(76) Inventor: Adreas F. Schaub, Walchwil (CH)

Correspondence Address: WENDEROTH, LIND & PONACK, L.L.P., 2033 K STREET N. W., SUITE 800, WASHINGTON, DC, 20006-1021, US

(21) Appl. No.: 10/501,984

(22) PCT Filed: Jan. 21, 2003
(86) PCT No.: PCT/EP03/00548

(30) Foreign Application Priority Data
Jan. 22, 2002 (CH) 012/02

(51) Int. Cl. A61K 33/42; A61K 31/22

(52) U.S. Cl. 424/601; 514/546

(57) ABSTRACT

The invention relates to the use of a physiologically acceptable organic substance for producing a composition which is free of alkali metal salts of metaphosphates and can be used by women as a lubricant during vaginal childbirth. The inventive composition eases and accelerates the childbirth. When mixed with pharmaceutical active ingredients, said composition can also, for example, prevent infection and relieve pain.

The present invention relates to the use of a physiologically acceptable organic substance for producing a composition containing no alkali metal salts of metaphosphates for use as lubricant in vaginal child birthing by women. The lubricant effect is in this case achieved by introducing the composition via the vagina into the birth canal and forming a layer on substantially the entire surface of the birth canal or parts of the surface of the birth canal.

Vaginal birthing of a child is a complex process and is determined by three substantial factors: the item to be delivered (fetus, amnion, placenta), the birth canal (consisting of a bony portion and a soft-tissue tube) and the birth forces. Various birth forces which promote or inhibit the vaginal birth of a human fetus are known from the scientific specialist literature. Birth-promoting forces are in this connection the contractions and the force of gravity, while birth-inhibiting forces are the stretching force of the mouth of the womb and of the birth canal. Birth of a human child is divided into 3 phases: the dilation period, the expulsion period and the placental period. The normal duration of labor in primiparas averages 12 hours, and in multiparas averages 8 hours. The reason for the shorter average duration of labor in multiparas compared with primiparas is the reduced stretching force of the birth canal, because in multiparas the soft-tissue tube [inner soft-tissue duct (uterine segment—cervical canal—attached soft-tissue duct (vagina and vulva)] is thinned by the preceding vaginal deliveries. The prevailing doctrine relating to the mechanics of labor in humans is accordingly that the stretching force of the birth canal (the force necessary to open, to stretch and to thin the birth canal) is to be regarded as the substantial force opposing to child birth (Dudenhausen, Schneider, Frauenheilkunde and Geburtshilfe, Verlag De Gruyter (1994), pages 113 to 121).

In veterinary medicine, the birthmechanical significance of the frictional force between the item to be delivered and the birth canal has been known for decades. Lubrication of the birth canal to reduce the frictional force is a standard method in veterinary obstetrics (Richter, Götze; Tiergeburtshilfe, 4th edition; Verlag Paul Parey; Rechtsfragen in der Tiergeburtshilfe, page 614), and lubricants for this purpose are commercially available. Relatively large volumes of lubricants can be employed in animal labor. This makes it possible to use liquid, aqueous compositions which serve as substitute for the lubricating amniotic fluid. It is not possible to use such large volumes in human vaginal delivery for lack of practicability.

A substantial difference between delivery in animals and delivery in humans is that the role of the amniotic fluid in human labor at term has no significant relevance in relation to lubrication of the birth canal and may on the contrary increase the resulting frictional forces. Amniotic fluid has, as an aqueous substance, little lubricant effect per se in humans. At present, vaginal deliveries in humans are mostly performed only with a vertex presentation, where the escape of amniotic fluid during delivery must be designated negligible owing to the sealing by the head. The vernix caseosa, the only lubricant substance with the item to be delivered, is mostly no longer present at the time of labor and anyway has only little effectiveness on the head. The use of amniotic fluid or substitute amniotic fluid for lubricating the birth canal before or during a vaginal delivery in humans is therefore not an appropriate measure for reducing the frictional forces and for easing vaginal child birthing in humans.

There is also some discussion about the significance of the frictional force between fetus and birth canal in humans as a force opposing to delivery. Thus, for example, Moolgaoker found in an investigation on forceps delivery that the success of forceps delivery is also substantially influenced by this frictional force [Moolgaoker et al., Obstet. Gynecol. 1979; 54(3), pages 299-309]. Cherkassky also recognized part of the significance of the frictional force during an assisted vaginal delivery and therefore proposed in a patent to reduce the frictional force of the birth canal during an assisted vaginal delivery by inserting plastic sheets into the vagina and thus to facilitate forceps delivery (see U.S. Pat. No. 4,602,623).

RU-C2-2177789 describes treating the cervix with a gel containing sodium hyaluronate for the induction of labor, in order to achieve softening and opening (ripening) of the cervix. Friction in the birth canal is not mentioned.

U.S. Pat. No. 3,614,797 proposes employing a composition of potassium metaphosphate, alginate, carboxymethylcellulose or carboxymethyl starch and sodium salts of weak acids in the form of a viscous and aqueous solution as substitute for amniotic fluid for lubricating the birth canal in mammals including humans, and, according to the disclosure, a lubricant effect is attributed to the amniotic fluid. However, it is known that water-based compositions display only a small effect in relation to lubrication. In addition, because of the content of metaphosphate salts, the composition is physiologically objectionable at least for humans.

It has additionally been known for a long time that, because of the lack of lubricity of the birth canal surface, a lubricant should be used for vaginal examination during the routine gynecological examination before delivery or during labor. The use of a lubricant in this case serves to facilitate manual vaginal examination and not to ease child birthing (Krantz K. E., Gorton, C. J., Obstet. Gynecol. 1973; 41(2), pages 308 to 309).

The technological advances in recent decades in obstetrics, in the care of pregnant women, in delivery management and in neonatology have made it possible to improve crucially infant and maternal morbidity and mortality. These advances have, however, also led to an increased rate of anesthetic procedures, vaginal surgical procedures and caesarian section, which in turn may lead to an increase in infantile and/or maternal morbidity.

In recent years, new delivery policies have been developed and introduced because of the legitimate demand of pregnant women for a gentle, technologically restrained delivery. These include mainly passive delivery management, restricted use of invasive methods and freedom of choice of the mode of delivery, such as waterbirth and alternative birthing positions. Although these policies have improved the childbirth experience with comparable morbidity and mortality rates, there is still a great and as yet unmet need by pregnant women for delivery, and especially the process of vaginal child birthing, to be facilitated and thus for the childbirth experience to be improved and for birth trauma such as damage to the pelvic floor to be reduced.

The birth-mechanical significance for delivery of the frictional force between the fetus and the birth canal in human childbirths has been overlooked until now. Effective reduction of this friction is unknown in human obstetrics. Nor have any pharmaceutical formulations for reducing friction in human obstetrics been developed, to say nothing of introduced.

It has now been realized, surprisingly, that human childbirth can be considerably facilitated and even shortened in duration, especially in primiparas, when the walls of the cervix and vagina (birth canal) are covered before and/or during labor with a physiologically acceptable organic lubricant. It is possible with the lubricant to reduce greatly the friction between birth canal and the item to be delivered both in the dilation period and in the expulsion period. In addition, the risk of injuries (such as, for example, thinning of the soft-tissue duct, damage to the pelvic floor, vaginal tears, perineal injuries, rectal injuries, uterine rupture, loss of blood) can be reduced or precluded, and long-term damage, such as, for example, urinary incontinence, fecal incontinence, sexual dysfunction and psychiatric disturbances can be limited or prevented. In addition, the work of labor can be reduced by the lower friction, possibly leading to a prevention or reduction of assisted vaginal deliveries or caesarean sections.

The invention relates to the use of a physiologically acceptable organic substance for producing a composition containing no alkali metal salts of metaphosphates for use as lubricant in vaginal child birthing by women.

The effect as lubricant is achieved for the purposes of the invention by introducing the composition to be used according to the invention into the birth canal. This forms a lubricant layer between preferably the entire surface of the birth canal and the fetus. Vaginal child birthing is considerably facilitated and expedited by a reduction in friction (irrespective of whether it is static friction, sliding friction and/or rolling friction) between fetus and surface of the birth canal.

Physiologically acceptable means for the purposes of the invention that the lubricants used are tolerated by the mother in labor and by the child.

Organic substance means for the purposes of the invention an organic substance with which lubricants can be produced.

Introduction of the composition to be used according to the invention advantageously takes place before the onset of labor and/or in the dilation period and/or in the expulsion period. It is possible in this way also to reduce friction of the unopened amniotic sac with the birth canal.

Many organic substances for producing lubricants are known and are already in use in medicine for a wide variety of purposes. The organic substances may themselves bring about the lubricant effect in the compositions (for example fats and oils which themselves have a lubricant effect), or they may confer a lubricant effect through the formulation of the composition (for example gels), it being possible for such formulations additionally to contain organic substances which themselves have a lubricant effect. The compositions may also form after introduction a lubricant film on the surface of the birth canal, which has a greater adhesion to the surface of the birth canal compared with the skin of the fetus.

The organic substances with a lubricant effect may be, for example, natural or synthetic oils, fats and waxes, such as, for example, fluid to viscous hydrocarbons or paraffins, vegetable oils and fats, hydrogenated or unhydrogenated fatty acid esters. Natural oils, fats and waxes may be of either vegetable or animal origin. Further possibilities are long-chain alcohols and esters thereof (fatty alcohols and esters thereof), polyols (glycerol or saccharides) and surfactants.

The organic substances with a lubricant effect may also be soluble, emulsifiable, dispersible, where appropriate low molecular weight, where appropriate biodegradable and/or where appropriate bioadhesive organic oligomers or polymers which can be formulated as compositions which form lubricant films or act as lubricants, for example with organic solvents, water or mixtures of solvents and water as vehicle. Examples of suitable solvents are ethers, alcohols and polyols, especially diols and triols, such as ethanol, ethylene glycol, propylene glycol, diethylene glycol, ethylene glycol monomethyl or dimethyl ether, ethylene glycol monoethyl or diethyl ether, diethylene glycol monomethyl or dimethyl ether, diethylene glycol monoethyl or diethyl ether, and glycerol. Esters such as, for example, ethylene carbonate and propylene carbonate are also suitable.

Organic substances which confer a lubricant effect through formulation of the composition are mainly polymers which can be swelled by organic solvents and/or water and which form gels. Organic substances which form hydrogels are preferred. It is also possible to employ more than one different polymer to form gels. Polymers for formulating gels may also have a lubricant effect themselves. Organic substances with a lubricant effect can also be added to gels.

The compositions with a lubricant effect may have an oily, waxy, pasty, mucilaginous or greasy consistency, or they may be gels, in particular hydrogels. However, emulsions or dispersions are also suitable. Examples of further forms of possible formulations are oils, pastes, creams, suppositories or foams. The mode of formulation has only an inconsiderable influence on the effect, but may influence the positive perception on use for the mother in labor.

The organic substances may be present alone or mixed with at least two substances. It is also expedient to formulate the substances to obtain forms which are simple to use, such as, for example, viscous oils, pastes, gels, creams, suppositories or foams, which can be distributed uniformly and completely on the surface of the entire birth canal. It is additionally possible with the formulation for the adhesion to the surface of the birth canal to be improved and, where appropriate, additionally to the fetus skin to be reduced, so that an optimal duration and lubricant effect of the lubricant layer applied to the surface of the birth canal is achieved.

The production of oils, pastes, gels, creams, suppositories or foams, and solutions, emulsions and dispersions for medical purposes and ingredients to be used for such formulations are known and need not be explained in detail here. Besides the lubricants, examples of aids which are employed are surfactants and dispersants, thickeners, pH buffers to adjust a physiological pH, solvents, carriers, fillers and preservatives. The organic substance may, depending on the activity, be present in the formulation in an amount of from 1 to 99% by weight, preferably 2 to 95% by weight, more preferably 5 to 90% by weight, and particularly preferably 5 to 80% by weight, based on the formulation.

Examples of suitable organic substances with a lubricant effect are:

a) edible vegetable and animal oils and fats, vegetable and animal waxes, mono- to polyesters of long-chain, saturated or unsaturated carboxylic acids and alcohols or polyols such as, for example, glycerol, pentaerythritol, trimethylolpropane or saccharides, fatty alcohols and carboxylic esters thereof, and mineral oils, mineral fats and mineral waxes (paraffins);

b) natural, natural and modified, or synthetic polymers such as, for example, cellulose, collagen, hydroxyalkylcelluloses (hydroxyethylcellulose, hydroxypropylcellulose), carboxymethylcellulose and alkali metal salts thereof, cellulose acetate, hydroxyalkyl starch (hydroxylethyl starch, hydroxypropyl starch), gelatin, dextran and hydroxyalkylated dextrans, alginates, polyesters of hydroxy carboxylic acids (polylactate, polyglycolate, polysorbate and copolymers of the corresponding monomers), aliphatic polycarbonates (polybutylene carbonate), polyalkylene oxides (polyethylene oxide, polypropylene oxide, polyethylene oxide/polypropylene oxide copolymers), polyvinyl alcohol and polyvinylpyrrolidone, hyaluronic acid or alkali metal salts thereof, poly(meth)acrylic acid, poly(meth)acrylic acid alkylesters, poly(meth)acrylic acid hydroxyalkylesters, polyacrylamides, polyvinyl esters (polyvinyl acetate), olefin/vinyl acetate copolymers, polyurethanes, polyamides, polysiloxanes (silicones), polyesters of dicarboxylic acids and diols, and polyethers.

Specific examples of organic substances as lubricants are: hydroxyethylcellulose, glycerol, polycarbophil, Carbopols (for example Carbopol 907, Carbomer 934P), hyaluronic acid and salts thereof, succinylated gelatin, liquid paraffin, white petrolatum, polyethylene glycols and polypropylene glycols and polyethylene/polypropylene copolymers (Pluronic 127), dimethicone, dimethiconol, cyclomethion, vegetable oils and fats, animal oils and fats, mineral oils and fats, surface-active substances (surfactants such as nonoxynol-9, phospholipids, pulmonary surfactants such as Lucinactant, Beractant, phospholipida e pulmone suis).

Mention should also be made of polymers which form a lubricant film after application, for example hydroxyethylcellulose, hydroxypropylcellulose, hydroxypropylmethylcellulose, polyvinyl alcohol, polyethylene glycols, polypropylene glycols and ethylene oxide/propylene oxide copolymers.

Organic substances which confer a lubricant effect through formulation of the composition are, for example: poly(meth)acrylic acid, poly(meth)acrylic acid hydroxyethyl esters, polyacrylic acid glyceryl esters, poly(meth)acrylamides, polyvinylpyrrolidone, carboxymethylcellulose, substances of human or animal origin such as, for example, constituents of vernix caseosa, of the amnion, constituents of the placenta, substances similar to pulmonary surfacetants (for example colfosceril palmitate, Lucinactant, Beractant, phospholipida e pulmone suis), and constituents of the synovial fluid and of the eye.

Further specific examples are polyacrylic acid, carboxymethylcellulose (MG 50 000 to 700 000, degree of substitution 0.5 to 1.5), polyvinylpyrrolidone (MG 5000 to 150 000), Carbopols in concentrations of from 0.25 to 5% by weight (polyacrylic acid) in combination with glycerol and NaCl, celluloses, especially in concentrations of from 1 to 3% by weight, and in combination with humectants and isotonisizing substances, carob flours, in particular in concentrations of from 0.5 to 3% by weight, and in combination with humectants and isotonicizing substances.

No (registered) product for the described indication, namely reduction of friction during vaginal child birthing by humans, is available commercially anywhere.

However, products which in some cases may be suitable for this indication, namely lubrication of the birth canal, although their effect is not optimized for the use according to the invention, are commercially available. These products are provided and used for rubbing into fingers for examination by palpation of body orifices (for example the vagina), improving the feeling of vaginal dryness, reducing the frictional forces during diagnostic intervention (bronchoscopy), and other indications. Examples of such products are:

a) from human medicine: Instillagel®, Endosgel®, K-Y®; K-Y® product family (such as KY Ultragel® and others), Replens®, Mucogyn®, Echovist®, Levovist®, LAM IPM™ Personal Lubricant®, CoLiquifilm®, Very Private™, Trojan™, VagiSil™, Wet®, Astroglide®, ID Millenium® and ID® Familie, Pre-Seed®, Eros™, Divine No. 9®, Probe®, Surgilube®, Lubrajel®, Surfaxin®, Curosurf®, Exosurf®, Survanta®, Gyne-Moistrin™, Ceylor Gel®;

b) from veterinary medicine: Vetagel®, Celagel®, Bovivet Gel®, Degragel®.

In a preferred embodiment, the composition to be used according to the invention may additionally comprise an active pharmaceutical ingredient or a combination of at least two active pharmaceutical ingredients which serve as medicaments for certain indications occurring during delivery, for example inhibition of labor or promotion of labor, alleviation of labor pain, and prevention of infections. The amount of active pharmaceutical ingredients can be for example from 0.0001 to 10% by weight, preferably 0.01 to 10% by weight, and particularly preferably 0.01 to 5% by weight, based on the composition.

Some examples of labor-inducing substances are oxytocin, dinoprostone, sulprostone, misoprostol, and hyaluronidase.

Some examples of labor-inhibiting substances are chondroitin sulfate, hexoprenaline, fenoterol, magnesium sulfate, atosiban, calcium antagonists, and nitroglycerin.

Some examples of pain-relieving substances are ethyl chloride, bupivacaine, Carbostesin, lidocaine, mepivacaine, Rapidocaine, Scandicaine, Solarcaine, Xylesin, and Xylocaine.

Some examples of disinfectants are antibacterial and antimicrobial substances such as quaternary ammonium compounds, povidone-iodine, and iodine and iodine-containing compounds.

It is particularly advantageous to admix antiviral substances for example to prevent transmission of herpes or HIV from the mother to the child, for example nucleoside analogs, nucleosidal reverse transcriptase inhibitors, non-nucleosidal reverse transcriptase inhibitors, and protease inhibitors.

It has also proved expedient to admix pulmonary surface-active substances pulmonary surfactants), with which the respiratory activity of the neonate after delivery can be facilitated, for example colfosceril palmitate, Lucinactant, Beractant, phospholipida e pulmone suis, perfluorocarbons. Substances which allows the cervix of the uterus to ripen, for example dinoprostone, sulprostone, misoprostol, and hyaluronic acid, may also be present.

The composition to be used according to the invention is advantageously present in a package, for example jars or, better, tubes, or it can be used as pessary. In addition, a vaginal applicator is preferably employed. If jars are used, the composition can be applied with fingers or spatulas to the surface of the birth canal. Tubes are more expedient, and the composition can be applied from them by means of pressure on the surface of the birth canal. The size of the package can be chosen so that the amount of the composition is sufficient for a single application. The tubes can be provided with an extension which substantially corresponds to the length of the birth canal and to the end of which the orifice for emergence of the composition is attached. The orifice for emergence is expediently designed, for example as round aperture, so that the composition can be completely and substantially uniformly dispersed on the surface of the birth canal.

Use of the composition to be used according to the invention is simple and is effective when the composition is applied before the onset of labor, in the dilation phase, or in the expulsion phase. Application can take place one or more times. Application shortly before or during the dilation phase may have the advantage that the tissue in the birth canal is softened, additionally facilitating delivery.

Another aspect of the invention is a method for easing vaginal child birthing by women, in which a composition comprising a physiologically acceptable organic lubricant and no alkali metal salts of metaphosphates is introduced in an effective amount into the birth canal of women.

Effective amount means for the purposes of the invention a volumetric quantity of from 1 to 500 ml, preferably 5 to 200 ml and particularly preferably 10 to 100 ml. The volumetric quantity may depend on the nature of the formulation.

The composition to be used according to the invention provides a number of advantages and improvements about which a skilled worker has not thought at all to date.

Primary improvements are, for example:
a reduction in the work of labor and/or in the duration of labor,
a reduction in the pain of labor,
a reduction in birth trauma for mother and child,
a reduction in the risk of peripartum and neonatal infection through combining the lubricant with antimicrobial substances.

Further improvements for the mother are, for example:
avoidance and/or reduction of injuries to the mother's birth canal (cervical tear, vaginal tear, perineal tear, labial tear, vulvar injuries),
prevention of damage to the pelvic floor,
prevention of urinary incontinence, fecal incontinence and psychological disturbances,
reduction in the episiotomy rate
reduction and/or avoidance of assisted vaginal deliveries or the caesarean section rate,
reduction in the episiotomy rate
reduction and/or avoidance of anesthetic interventions (local, redional anesthesia, sedation, inhalation anesthesia),

Further improvements for the child are, for example:
avoidance and/or reduction of intrapartum fetal hypoxia,
avoidance and/or reduction of fetal asphyxia during delivery and the sequelae thereof,
avoidance and/or reduction of injuries to the child during delivery, such as clavicle fracture, plexus lesions or cerebral hemorrhages
a reduction in neonatal respiratory distress syndrome through a combination of the lubricant with substances similar to pulmonary surfactant
a reduction in the rates of mother-to-child infection related to bacterial or viral infections.

It can generally be stated that the experience of childbirth is improved, and health costs can be reduced by preventing complications following delivery and long-term sequelae.

The following example explains the invention in more detail.

### EXAMPLE

It is known from the literature that the statistical duration of the dilation period in primiparas is 600 minutes, of the expulsion period is 120 minutes and the total time is thus 720 minutes (normal group).

The lubricant used with the consent of the mother in labor is the gel K-Y® Jelly supplied by Johnson & Johnson. This lubricant is employed for palpating examinations of the vagina in order to improve the lubricity of the examiner's fingers. The lubricant is not known and not optimized for the purpose used, namely easing delivery through introduction into the birth canal.

Eight primiparas are selected for an identical delivery team. The gel is used during the dilation period and the expulsion period in amounts such that as much as possible of the surface of the birth canal is always sufficiently covered with the gel (a total of about 80 g of gel, this amount is far higher than the amount used for vaginal examination during delivery). The amniotic sac of six mothers in labor bursts within 30 minutes before the end of the dilation period.

With an average birth weight of 3189±326 g, an average head circumference of 34±1.1 cm, and an average body length of 47.6±1.8 cm, the average duration of delivery is in total 336±184 minutes, and the significant shortening of the time for the duration of delivery is observed. The neonatal outcome parameters (APGAR, pH of the umbilical cord) are comparable with the normal group.

The subjective assessment of the process of birth by the delivery team and by the mother in labor is positive in all cases, although a non-optimized product was used.

1-11. (Cancel)

12. A method for easing vaginal child birthing in a woman, which comprises introducing a composition comprising a physiologically acceptable organic lubricant and no alkali metal salts of metaphosphates in an effective amount into the birth canal of the woman.

13. The method as claimed in claim 12, wherein an organic substance itself brings about a lubricant effect in the composition, or in that the organic substance confers a lubricant effect through formulation of the composition.

14. The method as claimed in claim 13, wherein the organic substance with a lubricant effect comprises a natural or synthetic oil, fat or wax.

15. The method as claimed in claim 13, wherein the organic substance with a lubricant effect comprises soluble, emulsifiable, dispersible, optionally low molecular weight, optionally biodegradable and/or optionally bioadhesive organic oligomer or polymer formulated as a composition optionally forming a lubricant film or acting as a lubricant.

16. The method as claimed in claim 13, wherein the organic substance which confers a lubricant effect through formulation of the composition comprises a polymer which can be swelled by an organic solvent and/or water and which form a gel.

17. The method as claimed in claim 12, wherein the composition is in the form of a liquid, paste, gel, cream, suppository or foam.

18. The method as claimed in claim 12, wherein the composition is in the form of a solution, emulsion, dispersion, oil or hydrogel.

19. The method as claimed in claim 12, wherein the organic lubricant is present in an amount of from 1 to 99% by weight in the composition.

20. The method as claimed in claim 12, wherein the composition further comprises an active pharmaceutical ingredient or a combination of at least two active pharmaceutical ingredients.

21. The method as claimed in claim 20, wherein the amount of active pharmaceutical ingredient or ingredients is from 0.0001 to 10% by weight, based on the composition.

22. The method as claimed in claim 20, wherein the active pharmaceutical ingredient or ingredients serve to inhibit child birthing or promote child birthing, alleviate pain, prevent infection, or prevent neonatal respiratory distress syndrome.
